(19) United States
(12) Patent Application Publication
(10) Pub. No.: US 2009/0043243 A1
(43) Pub. Date: Feb. 12, 2009

(54) METHODS AND DEVICES FOR LOADING TEMPORARY HEMOSTATIC SEALS

(76) Inventors: Paul A. DeLIPSKI, San Mateo, CA (US); Albert K. CHIN, Palo Alto, CA (US); Arthur M. LIN, Fremont, CA (US); Alfredo R. CANTU, Pleasanton, CA (US); Thang Tom UNG, Milpitas, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/113,484

(22) Filed: May 1, 2008

Related U.S. Application Data
(60) Provisional application No. 60/916,006, filed on May 4, 2007.

(51) Int. Cl. A61M 5/00

(52) U.S. Cl. 604/9

(57) ABSTRACT

Disclosed herein are methods, devices, and systems for folding a sealing element. A loader can receive a substantially unfolded sealing element, which when moved through the loader is configured for receipt in an insertion instrument. For example, the sealing element can be folded into a substantially prolate spheroidal shape. The folded sealing element can then be inserted into the insertion instrument. In one aspect, the loader is adapted to allow insertion of the folded sealing element into the insertion instrument when the folded sealing element is within a channel of the loader.

This application claims priority to Provisional Application Ser. No. 60/916,006 entitled “Methods And Devices For Loading Temporary Hemostatic Seals” filed May 4, 2007, which is incorporated herein by reference

### BACKGROUND

Contemporary coronary artery bypass grafting surgery is performed on a beating heart to reduce complications commonly associated with the prior surgical practice of transitioning a patient onto and off of a heart-lung machine. Performing an aortotomy and a proximal anastomisis on an aorta that is perfused with pressurized blood can be facilitated with temporary sealing methods to curtail blood flow through the aortic hole. Side-bite and surface-oriented clamping mechanisms have been used to diminish blood loss during such procedures, but such temporary occlusions can damage the endothelium and dislodge emboli that may migrate through the circulatory system. Alternative schemes for performing an aortotomy and limiting loss of blood include introducing a plug or seal at the site of the aortotomy, but such schemes commonly inhibit convenient and rapid completion of the graft anastomosis.

Resilient, flexible hemostatic seals, or sealing elements, have been developed to temporarily plug the aortic hole while the medical provider grafts a bypass vessel to the hole. Exemplary hemostatic seals are described in U.S. Pat. No. 6,814,743 and in U.S. Patent Application Publication Nos. 2006/0079915 and 2006/0206221. Exemplary commercial seals are those in the HEARTSTRING™ family of proximal seal systems (Boston Scientific; Natick, Mass., U.S.A.).

However, temporary seals like these require the seal portion to be manually folded into a taco-like shape and partially inserted into an introducer before insertion into the aortotomy. Unfortunately, this seal may be easily cracked during manipulation. Moreover, the seal cannot be packaged in a preloaded state into the delivery tool without increasing the risk that the seal will be cracked in transit or before implantation. Thus, a need remains for methods for loading seals into introducers, devices to practices those methods, and systems including the same.

### SUMMARY

Disclosed herein are methods and devices for folding sealing elements into a configuration that allows placement in an insertion instrument. In one aspect, loading a sealing element into an insertion instrument can include inserting a resilient, flexible sealing element into a loader comprising an elongate body having an inner channel. The proximal end of the elongate body can have an opening sized and shaped to receive the sealing element in an unfolded configuration. Moving the sealing element across at least a portion of the loader, causes the sealing element to fold.

In one embodiment, a loader device and system is disclosed. The system includes a hemostatic sealing element having a flexible body and a loader. The loader can comprise an elongate body extending between a proximal and distal end and having at least one opening sized and shaped for receipt of the hemostatic sealing element in a substantially unfolded configuration. In one aspect, the elongate body further comprising an inner channel that tapers in diameter along at least a portion of the longitudinal length of the channel, wherein the channel is adapted to fold the sealing element when the sealing element is moved through at least a portion of the channel.

In another embodiment, the loader can include an articulating folding feature. The articulating folding feature can be adapted to move between a first position and a second position. When moved from the first to the second position, at least a portion of the articulating folding feature moves relative to the channel and engages the sealing element. In one aspect, the articulating folding feature can apply pressure to the body of the sealing element. For example, the articulating folding feature can push one edge of the sealing element under a second edge of the sealing element to facilitate overlap of the first and second edges.

In one exemplary aspect, the articulating folding feature is a plunger. For example, the plunger can be movably mated with the elongate body. Moving the plunger from the first position to the second position can include moving the plunger transversely with respect to the elongate body. In another aspect, the articulating folding feature is not mated with the elongate body. For example, the loading system can further comprise a stylet for moving through an aperture in the sidewall of the elongate body.

The channel can have a shape configured to facilitate folding of the sealing element. In one aspect, the channel can have a taper. In another aspect, the channel can include a first portion having a constant diameter and second portion having a taper. In yet another aspect, at least a portion of the diameter of the inner channel includes asymmetric radii.

In another embodiment, the loader can be configured to orient the sealing element with respect to the articulating folding feature and/or with respect to an asymmetrically shaped inner surface of the elongate body. In one aspect, the at least one opening for receiving the sealing element can have a shape and size configured to allow ingress of the substantially unfolded sealing element in one orientation and to inhibit entry in a different orientation. For example, the at least one opening can be defined by a keyhole entry.

Another embodiment disclosed herein relates to a loader having diameter that a user can controllably vary. In one such aspect, the loader comprises an at least partially flexible body defining an elongate channel. The wall of the body can include a slit along at least a portion thereof. The body can have at least one opening for receiving a sealing element in a substantially unfolded configuration. A user can place the sealing element into the channel and compress at least a portion of the body to fold the seal. An insertion instrument can then be inserted into the at least one opening and the seal mated with the insertion instrument.

In another embodiment, methods of folding and/or loading the sealing element into an insertion instrument are disclosed. A method can include inserting a resilient, flexible sealing element into a loader comprising an elongate body having a distal end, a proximal end, and at least one opening. The elongate body can further comprise a channel shaped to fold the sealing element. An insertion instrument can be inserted into the at least one opening, the insertion instrument configured to received at least a portion of the sealing element in a folded position. Moving the resilient, flexible sealing element across at least a portion of the channel folds the sealing element. In one aspect, the sealing element is moved by pushing the sealing element through the channel with the insertion instrument.

Additional advantages will be set forth in part in the following description, and in part will be obvious from the description, or may be learned by practice of the invention. The advantages will be realized and attained by means of the elements and combinations particularly pointed out in the appended claims.

It is to be understood that both the foregoing general description and the following detailed description are exemplary and explanatory only and are not restrictive.

### BRIEF DESCRIPTION OF THE DRAWINGS

FIG. 1A is a cross-sectional view of one exemplary embodiment of a sealing element.

FIG. 1B is a top view of one exemplary embodiment of a system described herein.

FIG. 1C is a perspective view of the sealing element of FIG. 1A in a folded configuration.

FIG. 2A is a perspective view of one exemplary embodiment a sealing element loader.

FIG. 2B is a front view of another exemplary embodiment of the loader of FIG. 2A.

FIG. 2C is an exploded view of another exemplary embodiment of the loader of FIG. 2A.

FIG. 2D is a partially transparent perspective view of another exemplary embodiment of the loader of FIG. 2A.

FIG. 3 is a cross-sectional view of another embodiment of a loader described herein.

FIG. 4A is a cross-sectional view of one exemplary embodiment of a loader system described herein.

FIG. 4B is a cross-sectional view of the system of FIG. 4A with a sealing element positioned within the loader.

FIG. 4C is a cross-sectional view of the system of FIG. 4A with a folded sealing element within the loader.

FIG. 4D is a cross-sectional view of the system of FIG. 4A with the folded sealing element partially inserted into the insertion instrument.

FIG. 5A is a perspective view of an exemplary loader described herein.

FIG. 5B is a perspective view of another embodiment of the loader of FIG. 5A.

FIG. 5C is a perspective view of yet another embodiment of the loader of FIG. 5A.

FIG. 6A is a cross-sectional view of one exemplary embodiment of a loader.

FIG. 6B is a cross-sectional view of the loader of FIG. 6A with a sealing element positioned therein.

FIG. 7A is a perspective view of another exemplary embodiment of a loader described herein.

FIG. 7B is a partially transparent view of another embodiment of the loader of FIG. 7A.

FIG. 8A is a partially transparent side view of another exemplary embodiment of a loader system described herein.

FIG. 8B is a partially transparent side view of the loader system of FIG. 8A.

FIG. 8C is a partially transparent side view of the loader system of FIG. 8A.

FIG. 9A is a partially transparent perspective view of another exemplary embodiment of a loader described herein.

FIG. 9B is a front view of the loader of FIG. 9A.

FIG. 10A is an exploded view of another exemplary embodiment of a loader described herein.

FIG. 10B is a plan view of the loader of FIG. 10A.

FIG. 10C is a partially transparent view of the loader of FIG. 10A.

FIG. 11 is a perspective view of one exemplary embodiment of a loader described herein.

FIG. 12 is a partially transparent view of another embodiment of the loader of FIG. 11.

### DETAILED DESCRIPTION OF EXEMPLARY EMBODIMENTS

Reference will now be made in detail to the present embodiments, examples of which are illustrated in the accompanying drawings. Wherever possible, the same reference numbers will be used throughout the drawings to refer to the same or like parts.

Disclosed herein are methods, loader devices, and systems useful for loading temporary, hemostatic sealing elements into delivery tools. In turn, these sealing elements may be used to close aortic apertures. These methods, loaders, and systems reduce the risk of cracking the hemostatic seal while loading it into the delivery device.

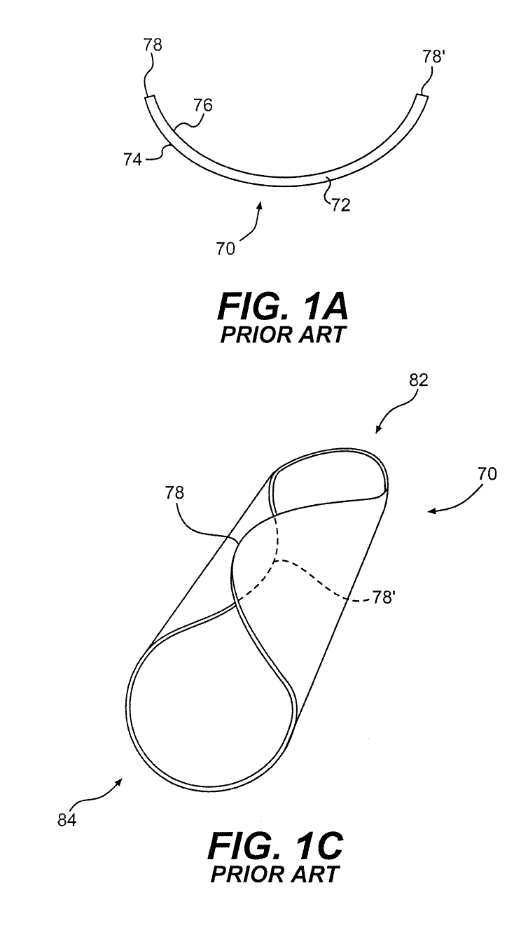

An unfolded sealing element 70 is depicted in FIGS. 1A and 1B. In order to insert sealing element 70 into an aortic aperture, sealing element 70 may be folded by overlapping any two of the seal's non-adjacent edges 78 and 78′. In one aspect, as illustrated in FIG. 1B, edges 78, 78′ are opposing edges, spaced by 180 degrees from one another. Alternatively, the edges can be spaced apart at least 90 degrees and in another aspect, spaced apart in the range of about 150 and about 180 degrees.

Sealing element 70 can represent the variety of hemostatic sealing elements. In one exemplary embodiment, sealing element 70 includes a body 72 having a “mushroom” shape with a concave lower surface 74 and a convex upper surface 76. The upper surface of seal body 76 can include a generally circular upper lip with the lower surface curving from the upper lip to a lower point or surface 80. However, one skilled in the art will appreciate that a variety of differently shaped sealing elements, can be used with the loader devices described herein.

Folding of sealing element 70, as illustrated in FIG. 1C, renders the previously mushroom-shaped sealing element 70 in the shape of an prolate spheroid or ellipsoid with somewhat blunted or flattened ends 82, 84. In latitudinal cross-section, the folded seal can be substantially circular; however, non-circular shapes, such as oval, rectangular, and triangular shapes can be achieved by varying shape of the loader channels described below. Thus, while the term “diameter” is used to describe a dimension of the sealing elements and/or loaders below, the loaders described herein are not limited to circular inner and/or outer cross-sectional shapes and the term “cross-sectional width” can be used interchangeably with diameter. Longitudinally, the cross-section appears generally elliptical with blunted or flattened ends 82, 84. The terms “prolate spheroid” and “prolate ellipsoid” are not used here in their precise geometric sense. Rather, a section of these shapes can describe a portion of the folded seal body 72 between ends 82, 84, where the folded sealing element has a generally taco or football-like shape. Once folded, sealing element 70 can be loaded into an insertion instrument 50.

Thus, in one aspect, methods for folding sealing element 70 into this folded configuration are disclosed. These methods can include moving the sealing element through a loader to reconfigure the shape of the sealing element. Once folded, the method can include positioning the sealing element, at least partly, in a delivery device. In another aspect, loaders are described for folding the sealing element when the sealing element is moved through a channel defined by at least a portion of the inner surface of the loader. For example, as described below, the loader can have a variety of differently shaped inner surfaces including a tapered inner surface, curved shape, and/or asymmetrical configuration.

In another aspect, exemplary insertion instruments are described, such as insertion instrument 50 illustrated in FIG. 1B. Insertion instrument 50 can include sheath 132 defining a lumen for received at least a portion of sealing element 70 in a folded configuration. Sheath 132 can include at least one opening, such as an opening at the distal-most end of insertion instrument 50, for receiving sealing element 70. The insertion instrument can further comprise a plunger 134 disposed within sheath 132 for selectively ejecting sealing element 70 from the insertion instrument lumen.

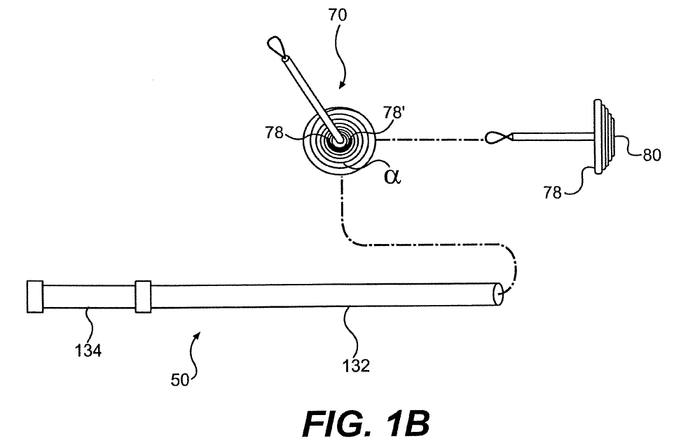

Referring now to the pictorial illustrations of FIGS. 2A-2D, a loader 10 for rendering sealing element 70 into a folded configuration is depicted. Loader 10 includes an elongate body 14 having a distal end 18 and a proximal end 19 with an opening 12 adapted to receive sealing element 70. The elongate body can define an inner channel 26 through which a sealing element is moved. As described below, the inner surface of the channel can have a shape configured to cause edges 78, 78′ to overlap and fold sealing element 70. In addition, elongate body 14 can receive a portion of an insertion instrument and permit loading of the sealing element into the insertion instrument while the sealing element is in a folded configuration.

Loader 10 can include, in one aspect, a tapered configuration, the size and shape of which moves edges 78, 78′ toward one another when a seal is moved through channel 26. However, as described below, alternative folding shapes and/or mechanisms can be used with the tapered shape to facilitate overlapping of edges 78, 78′.

In one aspect, at least a portion of the opening at proximal end 19 is defined by a proximal surface 21 that provides a keyhole entry. The keyhole entry includes an aperture having a shape corresponding, at least in part, to a cross-sectional shape of the sealing element in an unfolded configuration. To insert the sealing element, the unfolded sealing element is oriented to match the shape of the keyhole entry. In certain embodiments described below, the keyhole entry can align the sealing element with a non-uniform inner surface of channel 26 and/or with an actuating folding mechanism.

In addition, or alternatively, the keyhole entry can align the insertion instrument with channel 26, or more particularly, can align the insertion instrument with a seal after it has been folded inside channel 26. For example, the keyhole entry can include an aperture with a shape corresponding to a portion of the insertion instrument. Where a folded seal resides on the central longitudinal axis of the loader, the keyhole entry can provide a centrally located opening.

As depicted in FIGS. 2A through 2D, wall 21 can extend around a portion or all of the proximal end of body 14. Alternatively, as illustrated in FIG. 2B, wall 21 can a perimeter than corresponds with only a portion of body 14. In addition, the opening 12 defined by wall 21 can have two regions; a first region 13 for the passage of a sealing element and a second region 13′ for the passage of an insertion instrument. One such first region has a shape corresponding to an unfolded sealing element. For example, first region 13 can have a semi-circular shape. The second region 13′ can have a shape corresponding, at least in part, to the outer surface of the insertion instrument. For example, second region 13′ can have a circular shape. With respect to FIG. 2C, the first and second regions 13, 13′ overlap and together define the size and shape of opening 12. Alternatively, with respect to FIG. 2B, the first and section regions are not interconnected and define separate openings 12, 12′.

Proximal wall 21 can be integrated into elongate body 14 in one embodiment. In another embodiment, proximal surface wall 21 can be a distinct element that is fixedly or detachably mated with body 14. For example, FIG. 2C illustrates wall 21 including a mating member 15 that extends into channel 26 and has a shape corresponding to a portion of the inner surface of channel 26. Placing wall 21, including mating member 15, into a proximal portion of channel 26 can detachably mate the wall 21 and elongate body 14. In another aspect, the detachable wall can have an opening for passage of the sealing element, but not for the passage of the insertion instrument (e.g., either opening 12 is too small for the insertion instrument or does not allow proper alignment of the insertion instrument with a folded sealing element). A user can insert the sealing element through the keyhole entry and then remove wall 21 to insert the insertion instrument.

Referring to FIG. 2C, channel 26 of loader 10 can be formed of multiple elements. In one aspect, an insert 16 can be positioned within body 14 to define at least a portion of channel 26. For example, insert 16 can define a closed distal end. Alternatively, insert 16 can include an inner surface having a shape adapted to facilitate folding of the sealing element.

Once the loader folds, or at least partially folds, seal 70, the seal can then be at least partially loaded into sheath 132 of seal insertion instrument 50 by inserting the distal tip of the sheath into loader 10 and aligning the folded seal with the sheath. To assist with insertion of the seal into the insertion instrument, the loader can include an abutment surface 23 (FIG. 2D). The abutment surface can prevent further movement of the sealing element and/or act as a counter force when the sheath 132 is pushed against sealing element 70. For example, once the sealing element is positioned between the abutment surface and sheath 132, applying further force to sheath 132 can force the end of the folded seal into the distal tip of the sheath.

In one aspect, the folding feature does not fully fold the sealing element and/or the sealing element is folded (i.e., the sides are overlapped), but the diameter of the sealing element is larger than desired. Moving the sealing element into the sheath will reduce or compress the diameter of the sealing element and mate the sealing element and insertion instrument. Sheath 132 can then be withdrawn from the loader, while retaining the folded sealing element.

The user can also rotate insertion instrument 50 to facilitate loading. In one embodiment, insertion instrument 50 is rotated about one-quarter turn clockwise or counterclockwise. In another embodiment, insertion instrument 50 is rotated about one-half turn clockwise or counterclockwise. In another embodiment, insertion instrument 50 is rotated about a full turn or more clockwise or counterclockwise.

Distal end 18 of loader 10 is depicted illustratively as closed. But the skilled artisan understands that distal end 18 may be open. For example, where the distal end of the loader defines the abutment surface, the distal end of the loader can have an opening sized to prevent passage of the sealing element.

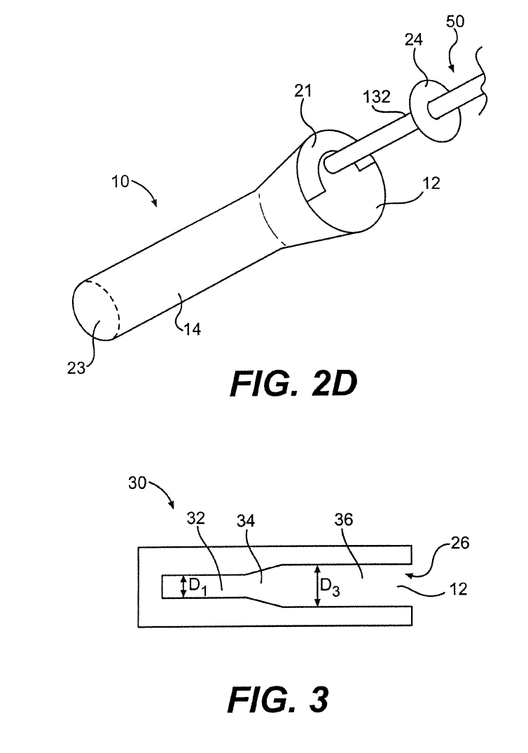

Loader 10 and/or insertion instrument 50 can include a hard stop to limit movement of the insertion instrument into channel 26. For example, where partial insertion of the sealing element into sheath 132 is desired, the insertion instrument can be prevented from insertion into channel 26 more than a predetermined distance. In one aspect, as illustrated in FIG. 2D, hard stop 24 is positioned on sheath 132. A distal portion of sheath 132 can be inserted through opening 12 until hard stop 24 contacts a portion of body 14 and further distal movement of insertion instrument 50 is inhibited. For example, hard stop 24 can contact a portion of wall 21. Other exemplary configurations of hard stop 24 are described with respect to FIGS. 4A-4D.

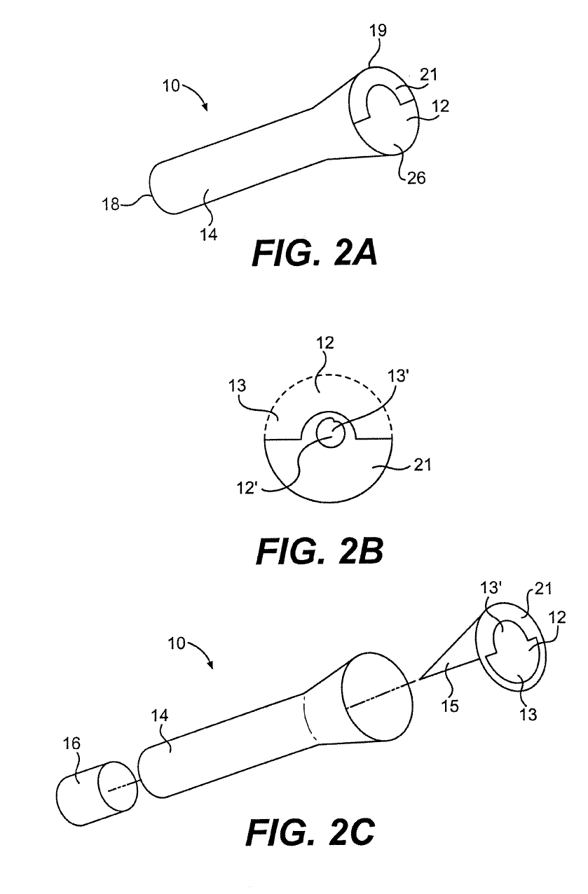

Referring now to loader 30 depicted in FIG. 3, loader 30 can include a channel wherein at least a portion of the diameter of the channel tapers. As shown, loader 30 may include at least three portions: (1) a first portion 32 with a first diameter D₁; (2) a second portion 34 with a tapering diameter; and (3) a third portion 36 with a third diameter D₃. The tapering diameter of second portion 34 may decrease from the third diameter to the first diameter in the longitudinal direction.

Sealing element 70 may enter loader 30 at proximal opening 12 and traverse at least a portion of the longitudinal length of loader 30. In one aspect, third diameter D₃ is larger than the largest cross-sectional width of an unfolded sealing element. In another aspect, placing the sealing element into the proximal end of channel 26, requires partial folding or bending of the sealing element. For example, the third diameter D₃ can be smaller than the largest cross-sectional width of the sealing element, but not small enough to require overlapping of sealing element edges.

As sealing element 70 traverses second portion 34 of loader 70, the decrease in diameter causes the edges of the sealing element to slide up the inside surface of the channel and take a shape corresponding to the inner surface of the channel. A further reduction in diameter cause the sealing element edges to overlap and the seal to fold. The first portion 32 can have a diameter D₁ smaller than the maximum outer diameter of a folded sealing element.

FIGS. 4A-4D depict a method for loading sealing element into insertion instrument 50 using loader 30. Sealing element 70 is attached to insertion instrument with suture line 52. As illustrated in FIG. 4B, sealing element can be partially folded to fit within the third portion 36 of channel 26. The insertion instrument is also inserted into loader 30 and can be used to push sealing element 70 through channel 26. As the sealing element is moved across the tapering portion 34, sealing element continues to be rolled into the folded configuration. In FIG. 4C the insertion element 50 pushes folded sealing element to first portion 32. Finally, in FIG. 4D, the insertion instrument forces folded sealing element 70 against abutment surface 23, forcing sealing element 70 at least partially into sheath 132 of insertion instrument 50.

In one embodiment, a portion of the insertion instrument and/or channel 26 can provide a hard stop to limit the distance that the folded sealing element is inserted into sheath 132. FIG. 4D illustrates insertion instrument 50 having a distal most end 133 smaller than the diameter of first portion 32 and a larger diameter portion 133′ of sheath 132 having a size greater than the diameter of first portion 32. When the larger diameter portion 133′ of the sheath reaches a portion of the channel having a diameter smaller than the larger diameter portion 133′, further distal movement of sheath 132 is prevented.

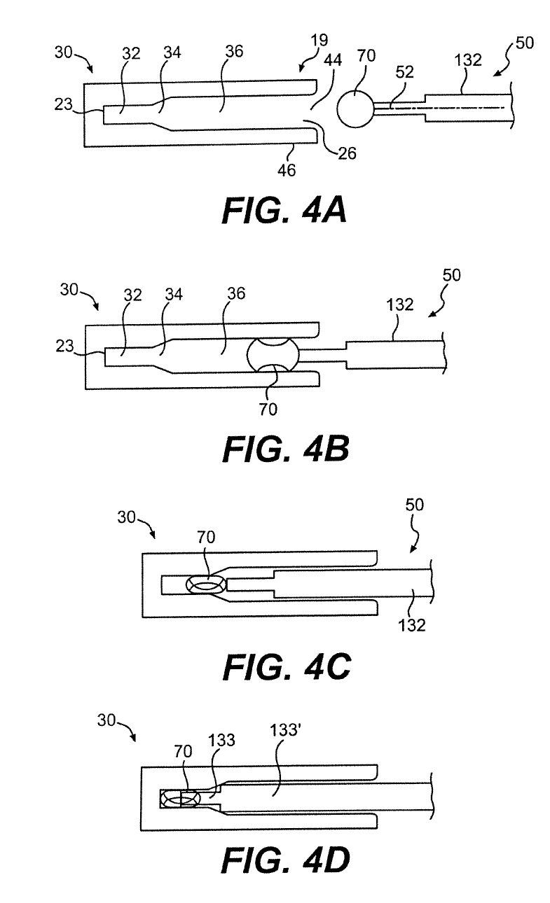

In another embodiment, a loader may include an articulating folding mechanism to assist with folding of the sealing element 70. For example, as depicted in FIGS. 5A and 5B, the loader 20 may include an aperture sized to receive a plunger 22. Plunger 22, in one aspect, can move relative to loader body 14 between a first position and a second position, where in the second position, the plunger extends into channel 26 of loader 20 and can contact sealing element 70. Upon contacting sealing element 70, the pressure applied by plunger 22 pushes a portion of sealing element 70 in a direction away from the plunger. This can initiate overlapping of the edges of the sealing element by moving a first edge of the sealing element away from the inner wall of the channel. Another edge of the sealing element can then slide over the first edge, facilitating overlap of the sealing element edges.

In one aspect, plunger 22 can be spring biased to the second position. Releasing the plunger applies an appropriate and predetermined force on sealing element 70. Alternatively, the plunger can be spring biased in the first position. A user can counteract the force of the spring bias to depress the plunger and move the plunger between the first and second positions.

Plunger 22 can be positioned to contact sealing element 70 closer to an outer edge. Thus, plunger 22 can initiate and/or assist with curling of one of the outer edges toward another, non-adjacent, outer edge. In one aspect, loader 20 includes a keyhole entry to align the sealing element with the location of plunger 22 when the sealing element is positioned in channel 26. The plunger therefore contacts a predetermined area of the sealing element, such as, for example, a portion of the sealing element adjacent to an outer edge.

Plunger 22, as illustrated in FIG. 5A, can be positioned along a tapered portion of body 14. The tapered shape of channel 26 and plunger 22 can work together to fold the sealing element. Methods for loading sealing element 70 using loader 20 can include inserting sealing element 70 into loader 20, moving sealing element 70 across loader 20 to plunger 22, depressing plunger 22, and further moving the sealing element within channel 26. In addition, a user can at least partially insert the insertion instrument into channel 26. In one aspect, the insertion instrument can push sealing element 70 across loader 20.

Alternatively, or additionally, plunger 22 as illustrated in FIG. 5B is located distally from the tapered section and can help to further fold a partially folded sealing element. For example, the plunger can contact a folded, or partially folded, sealing element to further reduce the diameter of the sealing element.

A variety of other articulating folding mechanisms can be used in addition, or as an alternative to the plunger. In one embodiment, the articulating folding mechanism is not mated with elongate body 14. Instead, as illustrated in FIG. 5C, loader 20 can have an orifice or aperture 130 for allowing access to sealing element 70. A stylet, finger, and/or other instrument can pass through aperture 130 to contact sealing element 70 and assist with folding.

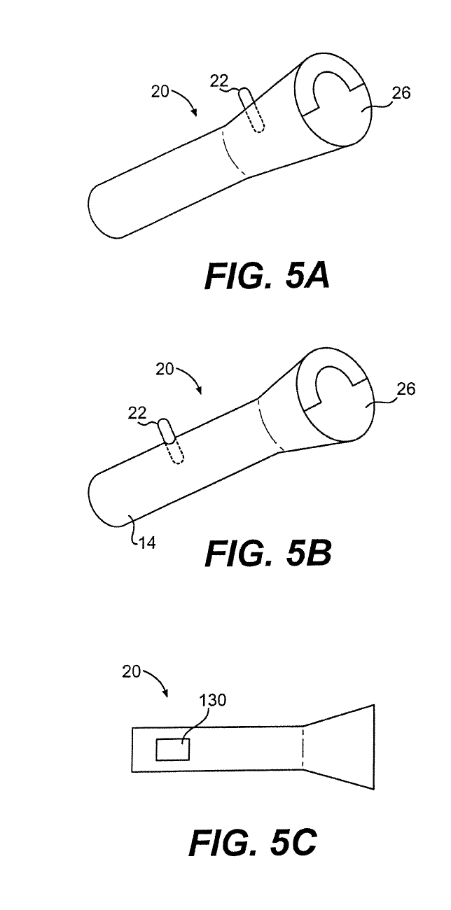

In another embodiment of the loader described herein, the loader can have folding features in addition, or as an alternative, to a tapered inner surface. A folding feature 38 is depicted in FIGS. 6A and 6B and includes a portion of loader 10 having an asymmetric diameter. This asymmetric diameter can comprise a first radius R1 and a second radius R2 in the inner channel 26.

The first radius can be sized to direct a first outer edge (e.g., outer edge 78) toward a second, spaced apart edge (e.g., outer edge 78′). The second radius similarly directs the second outer edge toward the first outer edge, but directs the second outer edge along a path such that the second outer edge overlaps the first outer edge. Explained another way, instead of directing outer edges 78, 78′ along corresponding pathways, the difference in the first and second radii facilitates an overlapping fold as the seal 70 moves through the loader 10.

In one aspect, the asymmetric folding feature is formed integrally with the loader. Alternatively, as illustrated in FIG. 2C, the folding feature can be defined by an insert 16 that is mated with the loader. Regardless, folding feature 38 can be positioned adjacent to the distal and/or proximal end of the loader. When folded sealing element reaches folding feature 38, movement of seal 70 into and/or through folding feature 38 at least partially loads sealing element 70.

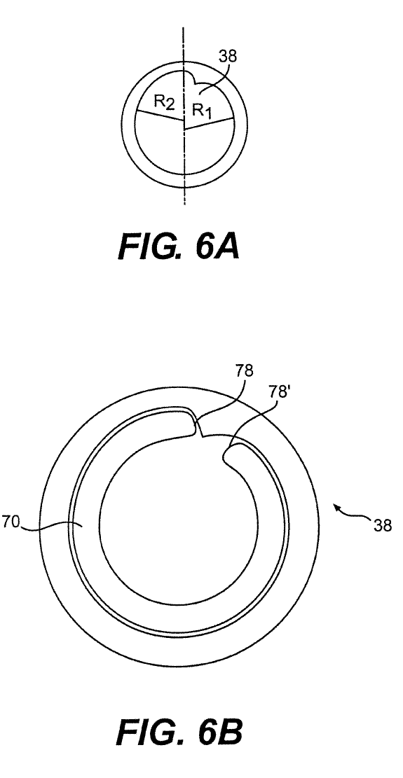

In another embodiment described herein, instead of the channel having a fixed, tapered diameter along its length, the inner diameter of the loader can be adjustable. As depicted in FIGS. 7A and 7B, loader 60 can have a slit 62 along at least a portion of its length. The material comprising loader body 64 can be a flexible and/or compressible material, allowing loader 60 to chance size when under pressure. For example, when squeezed, the width of the slit can be reduced to change the inner diameter of loader 60. In addition, or alternatively, the sides of the slit can overlap to allow a reduction in the diameter of the load body.

Prior to changing the diameter of loader body 64, the loader can have a tapered inner channel similar to the various embodiments of the loader described above. As shown in FIG. 7B, loader body can have an opening 66 at the proximal end and a closed distal end. Slit 62 can extend over a portion of body 64 to permit the proximal portion of the loader to compress. As the loader changes diameter, a sealing element positioned within loader 60 can fold. For example, opposing sides of the sealing element can be overlapped. A user can then insert insertion instrument 50 into proximal opening 66 and mate the sealing element and insertion instrument. In one aspect, the closed distal end of the loader can act as an abutment surface to facilitate insertion of the folded sealing element into the insertion instrument.

In another aspect, changing the inner diameter of the loader can partly fold or bend the sealing element. The insertion instrument 50 can then be introduced into the channel of the loader and used to move the sealing element further into the loader; causing the sealing element to fold. For example, after compressing the loader, the inner channel of the loader retains a tapered configuration and moving the sealing element through a portion of the tapered channel further folds the sealing element.

Alternatively, with respect to FIG. 7A, the inner diameter of loader 60 can have a generally constant width. Compressing the loader body 64 can substantially fold the sealing element. The insertion instrument can then be introduced into the loader and mated with the folded sealing element.

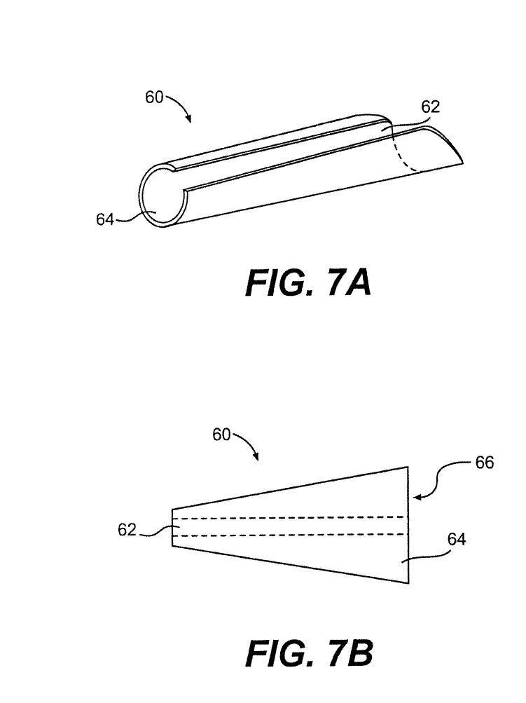

In another embodiment, the loader can be configured to mate with the insertion instrument. FIG. 8A illustrates a loader having first and second open ends 67, 69. Insertion instrument 50 can mate with first open end 67. For example, the interior surface of first open end 67 can have a size and shape corresponding to the outer surface of the distal-most end of the insertion instrument. In use, sealing element 70 can be pulled or pushed through a tapered portion of the loader to fold and/or deliver a folded sealing element into insertion instrument 50. One skilled in the art will appreciate that the loader and insertion instrument can mate in a variety of alternative ways, such as, for example, via a mechanical and/or frictional engagement.

In one aspect, as illustrated in FIGS. 8B and 8C, the loader is compressible. Sealing element 70 can mate with a suture line 52 which is passed through a slit (not illustrated) in loader 60 and extends into insertion instrument 50. Sealing element 70 can be placed in loader 60 and the loader can be compressed to fold or partially fold the sealing element. For example, with respect to FIG. 8B, the loader can be compressed such that the width of the loader moves from D₁ to a smaller width D₂. The sealing element can then be pushed and/or pulled through loader 60 to deliver the sealing element into the insertion instrument. In one aspect, the sealing element is fully folded by compressing loader 60, alternatively, after compression, the loader has a taper and pushing and/or pulling the sealing element through the loader completes the fold.

Once the folded sealing element is mated with the insertion instrument, loader 60 can be detached from the insertion instrument. For example, the loader can be pulled over the folded sealing element. Alternatively, in embodiments where the loader has a slit, a user can expand the slit to allow removal of the loader through the slit. Where the slit does not extend the full length of the loader or where no slit is present, the loader can have a break-away configuration. A user can tear or break the loader away from the insertion instrument.

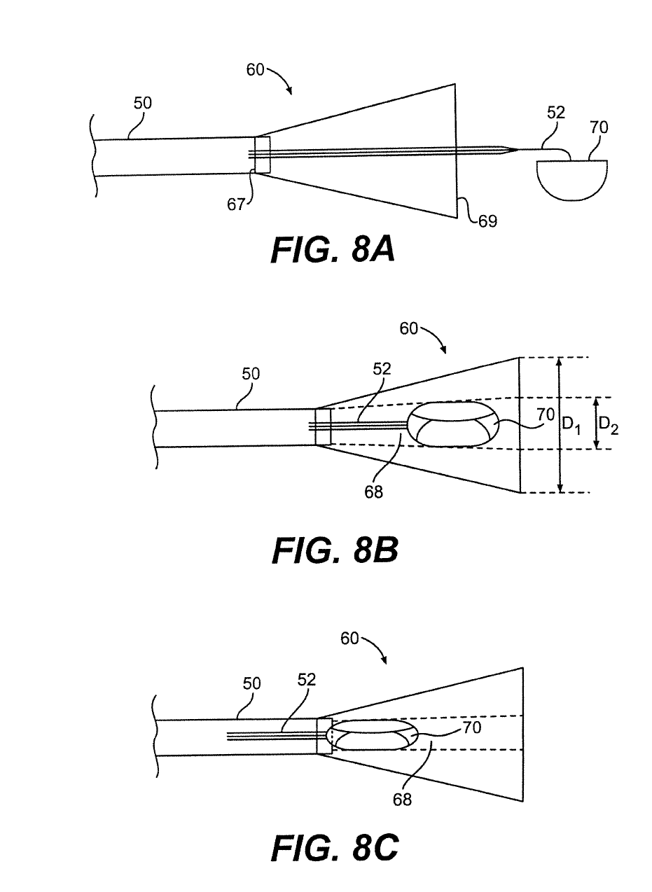

Another embodiment of a loader is depicted in FIGS. 9A and 9B. Loader 90 includes an elongate channel 94 having a varying cross-sectional width along its length. For example, the channel can taper along at least a portion of its length between an open proximal end and a closed distal end. In addition, the cross-sectional shape of the channel can vary along its length. In one aspect, the channel can have a cross-sectional shape defined at least in part by first and second segments 91, 93. The shape and/or size of the first and second segments can vary along the length of the channel.

In one aspect, as illustrated in FIG. 9B, first and second segments 91, 93 can be curved. Along the length of the channel, the radius of one of the curved first and second segments can vary at a different rate from the other. In use, one edge of the sealing element can be positioned adjacent to the first segment and another edge of the sealing element positioned adjacent to the second segment. As the sealing element moves through the channel, the difference rate of change of the radii of the first and second segments causes one edge of the sealing element to take a more severe curve. This can direct one edge of the sealing element under the other edge of the sealing element as the sealing element is moved through the channel.

Once folded, or partially folded, the sealing element can enter a portion of the channel where the first and second segments have the same shape. For example, the distal portion 95 of channel 94 can have a circular cross-sectional shape.

In order to align the sealing element such that the first and second segments correspond to different edges of the sealing element, the loader can include an alignment feature. In one aspect, as discussed above with respect to the keyhole entry, the shape of the proximal opening can matches a cross-sectional shape of the sealing element. In another aspect, as shown in FIG. 9B, the proximal opening 92 can have a notch 96 for receiving an edge of the sealing element. The notch can provide a visual indicator to a user of the proper alignment of the sealing element. In addition, or alternative, notch 96 can guide the sealing element into the loader channel in the proper alignment.

In another embodiment, the housing of the loader can be formed in two or more detachable pieces. The housing can be split or opened to expose at least a portion of channel 94. After folding the sealing element and inserting a portion of a folded sealing element into the insertion instrument, the housing can be opened to allow removal of the insertion instrument and sealing element. Instead of withdrawing the insertion instrument through the channel, the insertion instrument can be lifted out of the channel. In one aspect, this configuration can avoids rubbing the folded sealing element against the walls of the channel, and thereby reduces the risk of inadvertent or premature dislodging of the sealing element from the insertion instrument.

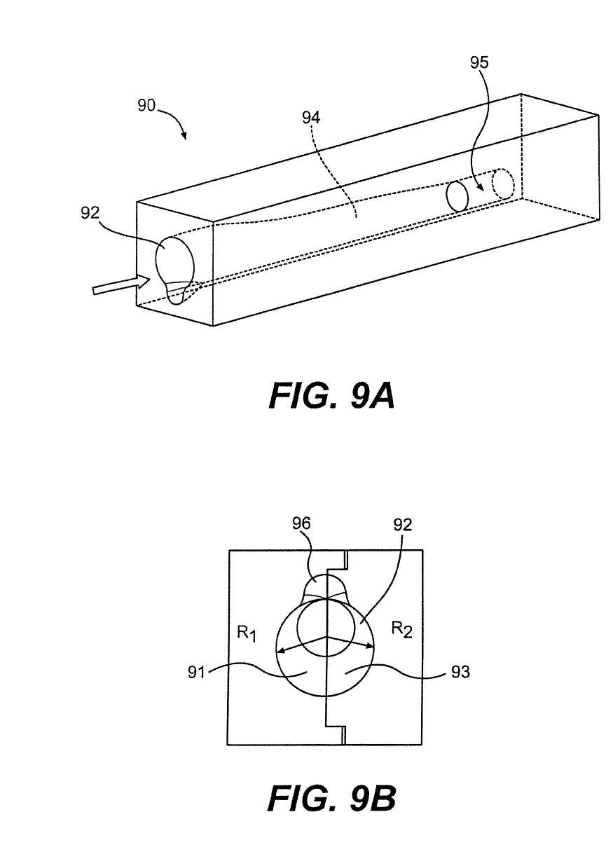

FIGS. 10A-10C illustrates such a loader 100. Cavities 106 and 106′ are recessed within a first body member 102 and a second body member 104, respectively. Body members 102, 104 are placed into contact with one another to form channel 126. Together, cavities 106, 106′ can define the shape of channel 126. In one aspect, the cavities can correspond to the first and second segments 91, 93 discussed above. For example, the radius of curvature of body cavity 106 may decrease more rapidly than cavity 106′.

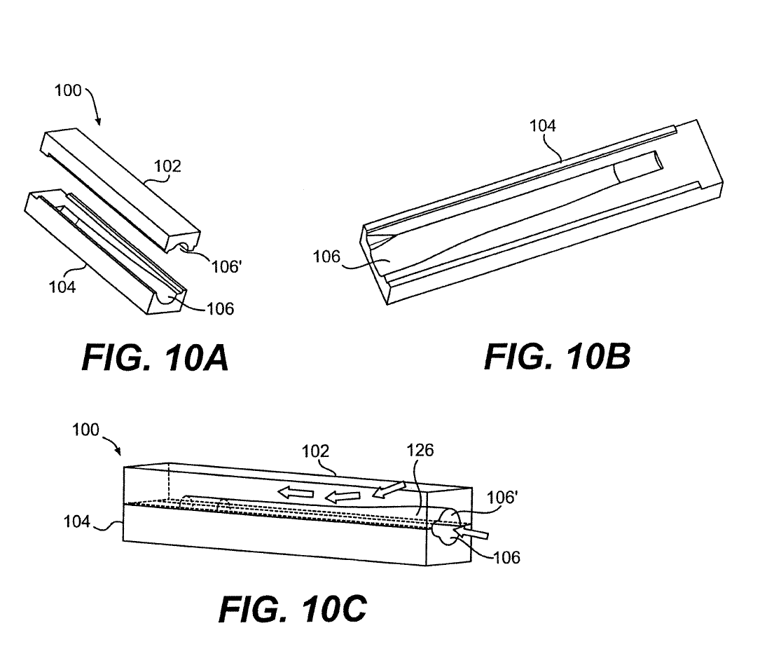

FIG. 11 depicts another embodiment of a loader comprising multiple body members that are movably mated with one another. Loader 110 depicts body members 112 and 114 joined together at their distal end with a hinge. Actuating the hinge can separate the body members and provide access to the channel. A variety of hinges can movably mate body members 112, 114, including, for example, living hinges, pin hinges, butt hinges, tee hinges, and combinations thereof.

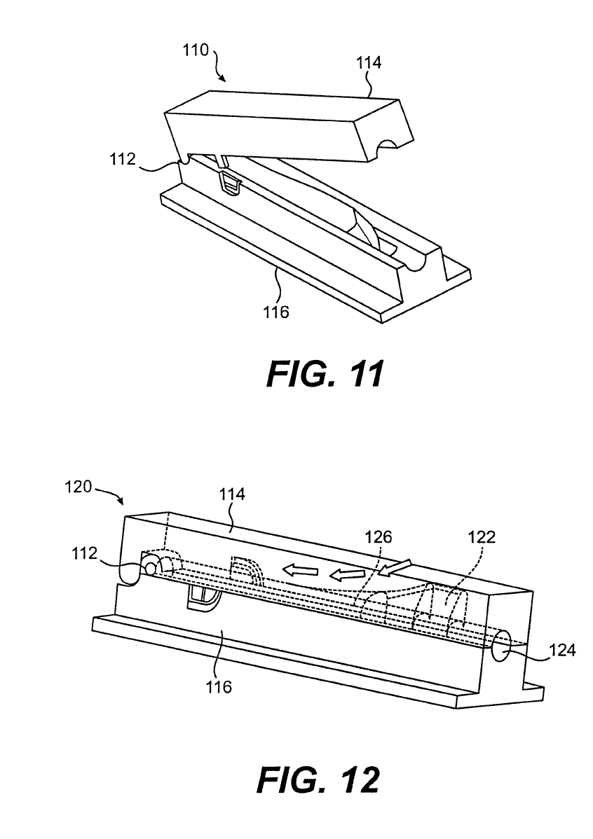

In yet another embodiment, the sealing element and insertion instrument can be inserted in the loader via different entrances. FIG. 12 depicts loader 120 having a channel comprising a first area 122 sized to accommodate an unfolded or partially folded sealing element 70 and a second area 126 having a reduced cross-section to fold sealing element 70. With the first area 122 holding a sealing element, an insertion instrument can enter loader 120 at opening 124 and push the sealing element into and/or through second area 126 to fold the seal element.

In one aspect, a user can open loader 120 to expose first area 122 and place a substantially unfolded sealing element therein. Loader 120 can then be closed by bringing body member 114 and 116 together. Opening 124 can have a sized and shape corresponding to a distal portion of an insertion instrument. For example, opening 124 can act align the insertion instrument with the seal positioned within first area 122. A user can direct the insertion instrument through opening 124 and move the sealing element through second area 126. As the sealing element moves through channel 126, loader 120 can fold the sealing element. Once the sealing element is folded, further insertion of the insertion instrument can mate the folded sealing element with the insertion instrument. A user can then removed the mated sealing element and insertion instrument by moving body member 114 away from body member 116 and exposing channel 126.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A loader system comprising:
a hemostatic sealing element having a flexible body;
a loader comprising an elongate body extending between a proximal and distal end and having at least one opening sized and shaped for receipt of the hemostatic sealing element in a substantially unfolded configuration, the elongate body further comprising an inner channel which tapers in diameter along at least a portion of the longitudinal length of the channel, wherein the channel is adapted to fold the sealing element when the sealing element is moved through at least a portion of the channel.

2. The loader system according to claim 1, wherein the at least one opening has a size and shape corresponding a cross-section of the sealing element body.

3. The loader system according to claim 2, wherein the at least one opening defines a keyhole entry that permits entry of the sealing element in a first orientation and inhibits entry of the sealing element in a second orientation.

4. The loader system according to claim 1, wherein the at least one opening is a keyhole entry comprising at least two interconnecting openings, wherein a first opening corresponds to an insertion instrument and wherein a second opening corresponds to the sealing element.

5. The loader system according to claim 1, comprising at least two openings, wherein a first opening has a size and shape corresponding to a size and shape of an insertion instrument and wherein a second opening has a size and shape corresponding to a cross-sectional size and shape of the sealing element.

6. The loader system according to claim 5, wherein the first opening is located on a central longitudinal axis of the elongate body.

7. The loader system according to claim 1, wherein at least a portion of the inner channel comprises a folding feature having an asymmetric diameter.

8. The loader system according to claim 1, wherein the inner channel comprises a first portion, wherein the diameter tapers, and a second portion, wherein the diameter is constant.

9. The loader system according to claim 1, wherein the diameter of the inner channel tapers from the proximal end to the distal end.

10. The loader system according to claim 1, further comprising an articulating folding feature extending through a sidewall of the elongate body.

11. The loader system according to claim 10, wherein the articulating folding feature is movably mated with the elongate body.

12. The loader system according to claim 11, wherein the articulating folding feature is adapted to move between a first position and a second position.

13. The loader system according to claim 12, wherein the articulating folding feature extends into the channel in the second position.

14. The loader system according to claim 12, wherein moving the articulating folding feature is adapted to apply pressure to the body of the sealing element when moved from the first to the second position.

15. The loader system according to claim 10, wherein the articulating folding feature is a plunger.

16. The loader system according to claim 1, wherein the channel is adapted to receive a portion of an insertion element having a lumen for receiving the sealing element in a folded configuration.

17. The loader system according to claim 16, wherein the channel includes a hard stop that prevents a distal end of the insertion instrument from entering a distal portion end of the channel.

18. The loader system according to claim 1, wherein the at least one opening is positioned proximate to the proximal end of the elongate body and the distal end of channel is closed.

19. The loader system according to claim 1, wherein the elongate body further comprises an aperture between the distal end and the proximal end, wherein the aperture is sized to receive an instrument configured to facilitate rolling of the sealing instrument.

20. The loader system according to claim 19, wherein the aperture is sized to receive a plunger.

21. The loader system according to claim 19, further comprising a stylet for insertion through the orifice.

22. The loader system according to claim 1, wherein the channel comprises at least three portions of different diameters, wherein at least one of the three portions has a constant diameter.

23. The loader system according to claim 1, wherein the elongate body is defined by first and second bodies that are movably mated to one another.

24. The loader system according to claim 23, wherein the first and second bodies are adapted to move relative to one another to expose the channel.

25. A loader useful for rolling a sealing element into a folded configuration comprising a flexible, hollow tube defining a channel for receiving the sealing element, a proximal end of the channel having a first inner diameter and an opening, a distal portion of the channel having a second, smaller inner diameter, the channel extending between the proximal opening and a closed end, wherein the tube comprises a slit along at least a portion thereof that allows variation of the first diameter of the channel.

26. The loader according to claim 25, wherein the slit extends from the proximal end to a point proximate to the closed end of the channel.

27. A method for loading a hemostatic seal into an introducer comprising:
inserting a resilient, flexible sealing element into a loader comprising an elongate body having a distal end, a proximal end and at least one opening, the elongate body comprising an at least partially tapered channel;
inserting an insertion instrument into the at least one opening, the insertion instrument configured to received at least a portion of the sealing element in a folded position; and
moving the resilient, flexible sealing element across at least a portion of the channel and folding the sealing element.

28. The method according to claim 27, wherein the moving step includes pushing the sealing element with the insertion instrument.

29. The method according to claim 27, further comprising the step of rotating the insertion instrument.

30. The method according to claim 27, wherein the at least one opening is a keyhole entry adapted to orient the sealing element relative to the channel.

31. The method according to claim 27, wherein at least a portion of the interior surface of the inner channel comprises a folding feature having an asymmetric diameter.

32. The method according to claim 27, wherein the inner channel comprises a first portion, wherein the diameter tapers, and a second portion, wherein the diameter is constant.

33. The method according to claim 27, wherein the step of moving folds the sealing element into a prolate spheroid shape.

34. A system of surgical components for anastomosing a graft vessel on a fluid-carrying vessel, comprising:
a sealing element including a continuous strand of material extending along a path forming an axial stem and a substantially lateral flange to a perimeter thereof;
an insertion instrument configured to receive at least a portion of the sealing element when the sealing element is at least partially folded; and
a loader configured to roll the sealing element into a folded configuration, said loader comprising a body having a distal end, a proximal end, and at least one opening for receiving the sealing element in a substantially unfolded configuration and for receiving at least a portion of the insertion instrument, the body further comprising a channel configured to fold the sealing element when the sealing element is moved through the channel.
